(19) United States
(12) Patent Application Publication
(10) Pub. No.: US 2026/0262921 A1
(43) Pub. Date: Sep. 10, 2026

(54) ENDOSCOPE VALVES

(71) Applicant: Boston Scientific Scimed, Inc., Maple Grove, MN (US)

(72) Inventors: Larry Edward Stanton, Burlington, MA (US); James J. Scutti, Norwell, MA (US); Colby Harris, Norfolk, MA (US); Robb Morse Gavalis, Westborough, MA (US); Allyn Narcisse Jensrud, Milford, MA (US)

(73) Assignee: Boston Scientific Scimed, Inc., Maple Grove, MN (US)

(21) Appl. No.: 19/561,059

(22) Filed: Mar. 9, 2026

Related U.S. Application Data
(60) Provisional application No. 63/769,310, filed on Mar. 10, 2025.

(51) Int. Cl. A61B 1/00; A61B 1/015

(52) U.S. Cl. CPC A61B 1/00068; A61B 1/00137; A61B 1/015

(57) ABSTRACT

The disclosure relates to endoscope valves. An example medical valve comprises a valve stem having a distal end, a proximal end, and a lumen extending along a longitudinal axis of the valve stem between a distal aperture and a proximal aperture; and a cap including a stationary portion and a movable portion which is movable relative to the stationary portion and fixed relative to the valve stem and is configured to move between a first configuration and a second configuration; and a lumen seal coupled to the valve stem, where the lumen seal is configured to: fluidically seal the distal aperture when the movable portion is in the first configuration; and permit fluid flow into the distal aperture when the movable portion is in the second configuration.

### CROSS-REFERENCE TO RELATED APPLICATIONS

This application claims the benefit of U.S. Provisional Patent Application Serial No. 63/769,310 filed on March 10, 2025, the disclosure of which is incorporated herein by reference.

### TECHNICAL FIELD

This disclosure relates generally to valve assemblies and methods, and particularly to endoscopes valves and methods of thereof.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems.

In an example, a medical valve is provided. The medical valve may include a valve stem having a distal end, a proximal end, and a lumen extending along a longitudinal axis of the valve stem between a distal aperture and a proximal aperture and a cap including: a stationary portion and a movable portion which is movable relative to the stationary portion and fixed relative to the valve stem and is configured to move between a first configuration and a second configuration, and a lumen seal coupled to the valve stem, wherein the lumen seal is configured to fluidically seal the distal aperture when the movable portion is in the first configuration and permit fluid flow into the distal aperture when the movable portion is in the second configuration.

Alternatively or additionally to any of the examples above, the lumen seal may be a flexible lumen seal that is configured to deform radially between the first configuration and the second configuration.

Alternatively or additionally to any of the examples above, the lumen seal may be an individual lumen seal.

Alternatively or additionally to any of the examples above, the lumen seal may be a one-way lumen seal.

Alternatively or additionally to any of the examples above, the lumen seal may have a radial diameter that is larger than a radial diameter of the distal aperture.

Alternatively or additionally to any of the examples above, the lumen seal may be a tapered lumen seal having a first diameter at a proximal end of the tapered lumen seal that is less than a second diameter at a distal end of the tapered lumen seal.

Alternatively or additionally to any of the examples above, a longitudinal axis of the lumen seal may be coplanar with a longitudinal axis of the lumen of the valve stem.

Alternatively or additionally to any of the examples above, the lumen seal may be a duck-bill check valve.

Alternatively or additionally to any of the examples above, the medical valve may include an actuation mechanism that is configured to contact the lumen seal to cause the lumen seal to permit fluid flow into the distal aperture when the cap is in the second configuration.

Alternatively or additionally to any of the examples above, the actuation mechanism may be an individual substantially longitudinally extending protrusion.

Alternatively or additionally to any of the examples above, the actuation mechanism may extend from a portion of an inner surface of the medical valve that is distal to the distal end of the valve stem.

Alternatively or additionally to any of the examples above, the actuation mechanism may be a substantially cylindrical actuation mechanism.

Alternatively or additionally to any of the examples above, the actuation mechanism may have a rounded proximal end.

Alternatively or additionally to any of the examples above, the medical device may include a first sealing member disposed circumferentially around the valve stem, wherein the first sealing member is configured to be disposed distal to an air inlet and an air outlet and is proximal to liquid outlet and a liquid inlet when the movable portion is in the first configuration and the second configuration.

Alternatively or additionally to any of the examples above, the medical device may include a second sealing member disposed circumferentially around the valve stem, wherein the second sealing member is configured to be disposed distal to an air inlet, an air outlet, and a liquid outlet and be disposed proximal to a liquid inlet of the medical valve when the movable portion is in each of the first configuration and the second configuration.

In another example, a medical valve is provided. The medical valve comprises a valve stem having a distal end, a proximal end, and a lumen extending along a longitudinal axis of the valve stem between a distal aperture and a proximal aperture, wherein the distal aperture is located at the distal end of the valve stem, a cap including: a stationary portion; a movable portion which is movable relative to the stationary portion and fixed relative to the valve stem, wherein the movable portion is configured to move the valve stem between a first configuration and a second configuration, a seal disposed between the stationary portion and the movable portion; and a biasing member configured to bias the movable portion to the first configuration; and a lumen seal coupled to the distal end of the valve stem, wherein the lumen seal is configured to: fluidically seal the distal aperture when the movable portion is in the first configuration and permit fluid flow into the distal aperture when the movable portion is in the second configuration.

Alternatively or additionally to any of the examples above, the medical device may include an actuation mechanism that extends substantially longitudinally from a portion of an inner surface of the medical valve that is distal to the distal end of the valve stem.

Alternatively or additionally to any of the examples above, the medical device may include a first sealing member disposed circumferentially around the valve stem, wherein the first sealing member is configured to be disposed distal to an air inlet and an air outlet and is proximal to liquid outlet and a liquid inlet when the movable portion is in the first configuration and the second configuration; and a second sealing member disposed circumferentially around the valve stem, wherein the second sealing member is configured to be disposed distal to the air inlet, the air outlet, and the liquid outlet and proximal to the liquid inlet of the medical valve when the movable portion is in each of the first configuration and the second configuration.

In another example a medical valve is provided. The medical valve comprises a valve body, a valve stem disposed in the valve body, the valve stem having a distal end, a proximal end, and a lumen extending along a longitudinal axis of the valve stem between a distal aperture and a proximal aperture, wherein the distal aperture is located at the distal end of the valve stem, wherein the valve stem is configured to translate within the valve body; and a cap including a stationary portion, a movable portion which is movable relative to the stationary portion and fixed relative to the valve stem, wherein the movable portion is configured to move between a first configuration and a second configuration; a seal disposed between the stationary portion and the movable portion, and a biasing member configured to bias the movable portion to the first configuration, a lumen seal coupled to the valve stem, wherein the lumen seal is configured to: fluidically seal the distal aperture when the movable portion is in the first configuration and permit fluid flow into the distal aperture when the movable portion is in the second configuration, and an actuation mechanism that is configured to contact a distal surface of the lumen seal to cause the lumen seal to deform and permit the fluid flow into the distal aperture when the movable portion is in the second configuration.

Alternatively or additionally to any of the examples above, the actuation mechanism may be configured to be spaced a distance away from the distal surface of the lumen seal when the movable portion is in the first configuration.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 depicts a schematic view of components of an illustrative endoscope;

FIG. 2 depicts a schematic view of components of an illustrative endoscope system;

FIGS. 3A-3B show cross-sectional views of an example of an endoscope valve in a first configuration and a second configuration, respectively; and

FIGS. 4A-4B show views of a distal end of the endoscope valve in the first configuration and the second configuration, respectively.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example configurations of the disclosure.

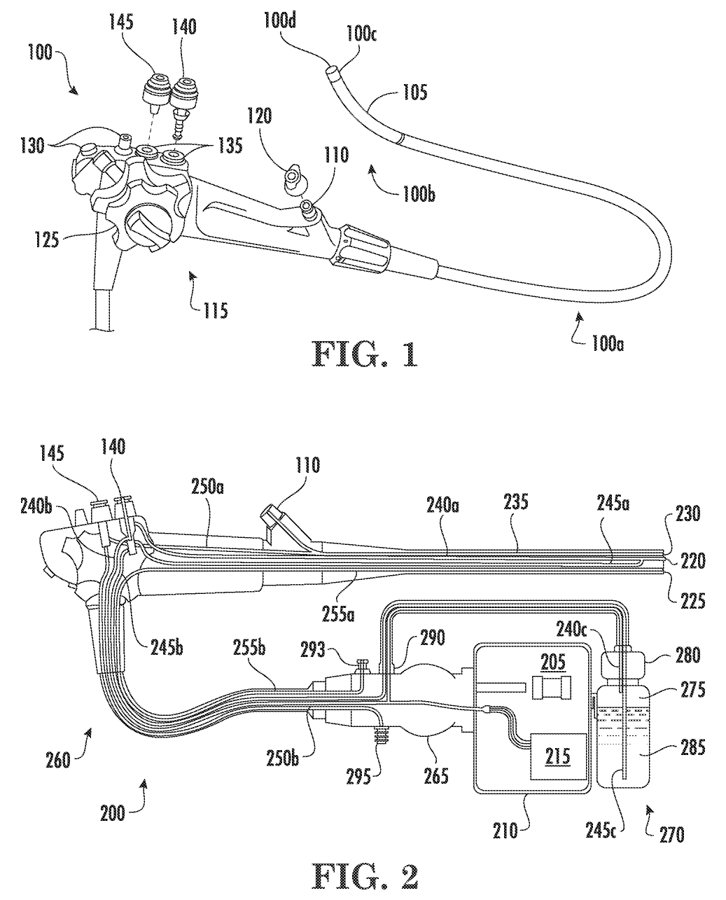

With reference to FIG. 1, an illustrative endoscope 100 is schematically depicted and FIG. 2 schematically depicts an illustrative endoscope system 200. Endoscope 100 may include an elongated tube or shaft 100a that is configured to be inserted into a subject (e.g., a patient).

A light source 205 of endoscope system 200 may feed illumination light to a distal portion 100b of endoscope 100. The distal portion 100b of endoscope 100 may house an imager (e.g., CCD or CMOS imager) (not shown). Light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump (e.g., an air pump 215), such as an air feed pump, in video processing unit 210.

Endoscope shaft 100a may include a distal tip 100c (e.g., a distal tip unit adapted to be inserted into a body cavity of a patient) provided at distal portion 100b of shaft 100a and a flexible bending portion 105 proximal to distal tip 100c. Flexible bending portion 105 may include an articulation joint (not shown) to assist with steering distal tip 100c. On an end face 100d of distal tip 100c of endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along shaft 100a for passing tools to the treatment area, may also be included on the face 100d of the distal tip 100c. Working channel 235 may extend along shaft 100a to a proximal channel opening 110 positioned distal to an operating handle 115 (e.g., a proximal handle) of endoscope 100. A biopsy valve 120 may be utilized to seal channel opening 110 against unwanted fluid egress.

Operating handle 115 may be provided with knobs 125 for providing remote 4-way steering of the distal tip via wires connected to the articulation joint in the flexible bending portion 105 (e.g., one knob controls up-down steering and another knob controls left-right steering). One or more (e.g., one, two, a plurality, etc.) of video switches 130 for remotely operating the video processing unit 210 may be arranged on a proximal end side of the handle 115.

Handle 115 may be provided with dual valve locations 135. One of the valve locations 135 may have or receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along shaft 100a to a junction point in fluid communication with the working channel 235 of the endoscope 100.

The operating handle 115 may be electrically and fluidly connected to video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects air pump 215 to the gas feed line 240b in the umbilical 260.

A water container or reservoir 270 (e.g., a water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other configurations, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water is supplied via a pump (e.g., peristaltic pump) from a water source independent (not shown) from the water reservoir 270. In other configurations, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

Referring to FIG. 2, an illustrative operation of an endoscope system 200, including an endoscope such as the endoscope 100 depicted in FIG. 1, is explained. Air from the air pump 215 in the video processing unit 210 may flow through the connector portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user’s finger on the valve, air is allowed to flow out of the valve 140 to atmosphere. In a first position, the user’s finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some configurations, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user’s field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve 145 may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position).

Endoscopes include functionality to deliver fluids (including air and water) and suction to a site of a procedure. Tubing for delivering fluids and/or suction extends from a handle of the endoscope, through a sheath of the endoscope, and to a distal tip of the endoscope. During a procedure, body fluids, tissues, or other material can build up in the tubing and, in some cases, lead to clogging of the tubing. In order to aid in reprocessing of reusable endoscopes between procedures, pre-processing is performed in an endoscopy suite. For example, water or other fluids are flushed through the tubing after the endoscope is removed from a patient, in order to clear debris from the air/water and/or suction tubing. One option for accomplishing such pre-processing is a reusable cleaning valve. The cleaning valve may be inserted into an air/water valve cylinder of an endoscope after the scope is removed from a patient. An operator may then depress a button of the cleaning valve for a predetermined amount of time (e.g., 30 seconds) to flush the air and/or water channels of the endoscope prior to further reprocessing of the endoscope. Such cleaning may require active intervention by an operator. A reusable cleaning valve must be subject to cleaning, itself, in between uses, which can add to reprocessing cost. Therefore, a need exists for valves capable of performing cleaning functions.

For instance, some previous approaches may seek to use endoscope valves or valve assemblies (e.g., removable or non-removable valve assemblies) that include one or more components (e.g., a valve stem) that rotates (e.g., responsive to actuation by an operator) to alter a configuration of a fluid/air pathway in the endoscope valve and thereby selectively permit water or other fluids are flushed through the tubing after the endoscope is removed from a patient (e.g., in an effort to clear debris from the air/water and/or suction tubing of the endoscope). However, such approaches may not readily provide tactical feedback related to actuation or initiation of a cleaning function (e.g., to ensure that a lumen in the valve stem is properly aligned or otherwise configured to receive water at an intended flow rate/volume to facilitate proper cleaning of the endoscope). Additionally, such approaches may be prone to various components (e.g., a distal end of the valve stem) incurring an undue amount of wear, for instance, due to repeated physical contact of the components with a hard surface such as a surface of the valve body and/or rotation of the components about a longitudinal axis of the valve. Such component wear, particularly in the absence of a lumen seal (e.g., a lumen seal at a distal end of the valve stem) may cause such previous valves to be prone to unintended leakage of air/water.

Hence, as detailed herein an endoscope valve may be configured to provide a cleaning functionality to an air channel of an endoscope and may be configured to mitigate unintended leakage of air/water, mitigate component wear, and/or provide tactile feedback. For instance, the endoscope valves herein can include a lumen seal located at or forming a distal end of a valve stem. The lumen seal can be configured to selectively permit a fluid (water) to flow into the lumen of the valve stem. In some configurations, the endoscope valve may be appropriate for a single-use and therefore be disposable. In a first configuration, the endoscope valve may provide air (e.g., only air) to an air channel (e.g., an air outlet) of an endoscope. In a second configuration, the endoscope valve may provide only water flow to only an air channel of the endoscope. The endoscope valve (hereinafter, valve) may include features that, after the valve is transitioned from the first configuration to the second configuration, retain the valve in the second configuration until an input is received (e.g., until a user actuates a button or movable portion of the valve) or may be configured to retain the valve in the second configuration for a predetermined amount of time, such as a time specified for flushing an air valve in a cleaning protocol. Thus, in some configurations the valve may be in a second, flushing configuration for a predetermined amount of time without active participation by a user, so that the user may perform other tasks during the flushing of the air channel of the endoscope. After the predetermined amount of time, the valve may transition from the second configuration back to the first configuration automatically. However, in some configurations, the valve can include various components (e.g., a cam, a plunger, etc.) to provide the valve with a click-pen type functionality. For instance, a first click or actuation of the cap may move the valve from the first configuration to the second, flushing configuration and a second click or actuation of the cap transitions the valve from the second configuration back to the first configuration. Alternatively, in some configurations, the valve can include one or more threaded elements (e.g., a threaded valve stem) which are configured to permit the valve to be rotated and thereby cause the valve to transition between the first configuration and the second, flushing configuration.

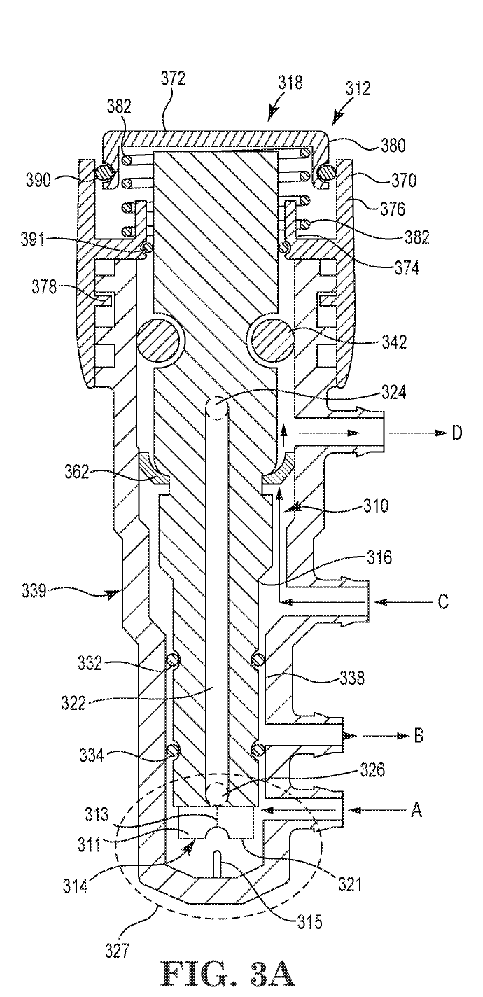

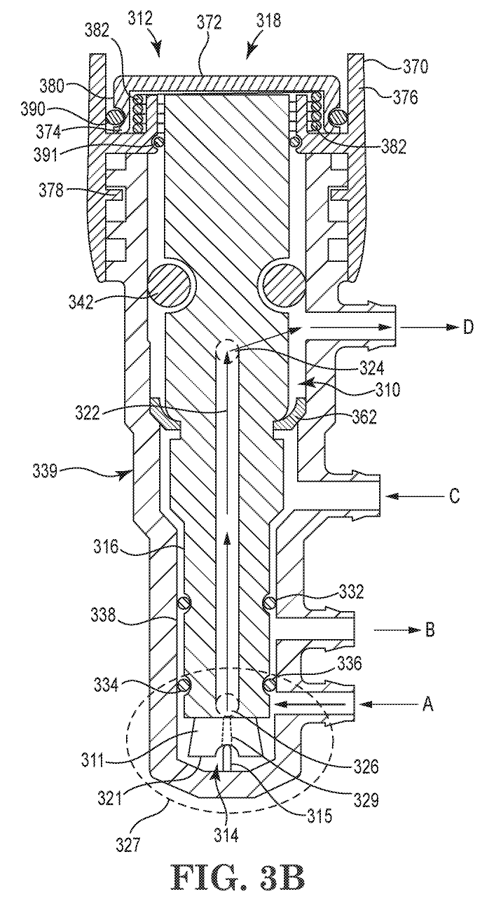

FIGS. 3A and 3B show cross-sectional views of an illustrative configuration of a cleaning valve 310 (e.g., a medical valve) in a valve cylinder 339. FIG. 3A shows valve 310 in a first configuration, and FIG. 3B shows valve 310 in a second, flushing configuration. Valve cylinder 339 may have a water inlet A, a water outlet B, an air inlet C, and an air outlet D. Water inlet A may be in fluid communication with a source of water or other liquid (e.g., water, cleaning solution, air, other gases, or combinations thereof). Water outlet B may be in fluid communication with a water channel of an endoscope (not shown), which may extend from a proximal end of the endoscope to a distal end of the endoscope. During a medical procedure, the water channel may be used to deliver water at a site of the procedure. Air inlet C may be in fluid communication with a source of air or other fluid (e.g., air, other gases, water, or cleaning solution, or combinations thereof). Air outlet D may be in fluid communication with an air channel of the endoscope. During a medical procedure, the air channel may be used to deliver air at a site of the procedure.

Valve 310 may have a proximal end 312 and a distal end 314. A valve stem 316 may extend from proximal end 312 to distal end 314. A cap 318 (which may be an operation portion of valve 310) may be disposed at proximal end 312. Valve stem 316 may be a single, unitary structure formed of a single, continuous piece of material and may be made from a metal (e.g., stainless steel, titanium, aluminum, etc.), from a polymer (e.g. polycarbonate, ABS, HDPE, Nylon, PEEK, thermoplastic, plastic, etc.), or from any other suitable material. Depending on the material used, valve stem 316 may be machined, injection molded, extruded (via, e.g., 3D printing), or otherwise formed. Valve stem 316 may be formed of a clear thermoplastic so that certain portions of an interior of valve stem 316 are visible through external walls of inner cylindrical member.

Valve stem 316 may have a lumen 322 extending through a central longitudinal axis of valve stem 316. Alternatively, lumen 322 may extend through another longitudinal axis of valve stem 316 (e.g., lumen 322 may be off-centered). A space between an exterior surface of valve stem 316 and a surface defining lumen 322 may be solid, and lumen 322 may be a bore formed in valve stem 316. In another example, a space between an exterior surface of valve stem 316 and a surface defining lumen 322 may be hollow. In such a case, lumen 322 may be formed by a longitudinal tube within valve stem 316.

Lumen 322 may be open to an exterior of valve stem 316 on a proximal end of lumen 322 via one or more proximal apertures 324. For example, lumen 322 may be fluidly connected to proximal aperture(s) 324 via a second, proximal lumen (not shown) which may be transverse to lumen 322. For example, the second lumen may be perpendicular to lumen 322 (extending into the page in FIG. 3A). Lumen 322 may be open to an area exterior of valve stem 316 on a distal end of lumen 322 via one or more distal apertures such as a distal aperture 326. Lumen 322 may be fluidly connected to distal aperture(s) 326. The distal aperture(s) may be located at or otherwise provide an opening in or near the distal end of the valve stem 316, as illustrated in FIGS. 3A-3B. The distal aperture 326 can be coplanar or positioned along a same longitudinal axis as the lumen 322. Proximal aperture 324 may be disposed axially between one-way seal 362 and proximal seal 342. Distal aperture 326 may be disposed axially between second distal seal 334 and a distal end of the valve stem 316. In some examples, the distal aperture 326 may be co-axial with a central axis of the lumen 322 and extend through the distal end of the valve stem 316.

Valve stem 316 may have disposed on it a first distal seal 332 and a second distal seal 334. Distal seals 332, 334 may be made from elastomeric material. Distal seals 332, 334 may be identical to one another and may be, for example, O-rings. Distal seals 332, 334 may be disposed in circumferential, annular grooves or indentations on valve stem 316. A durometer value and outer diameter of distal seals 332, 334 may be such that the distal seals 332, 334 have an interference fit with an inner surface 338 of an endoscope valve cylinder 339 when valve 310 is inserted in endoscope valve cylinder 339. The interference fit may be loose enough so that valve stem 316 may slidably move relative to surface 338 but tight enough so that fluids cannot flow longitudinally between a radially outermost surface of seals 332, 334 and surface 338. Second distal seal 334 may be proximal to distal aperture 326. Second seal 334 may be distal to the water outlet B and proximal to the water inlet C. For instance, the second seal 334 may be distal to the water outlet B and proximal to the water inlet C when the movable portion is in both the first configuration and the second configuration (e.g., when the lumen seal is configured in a closed configuration and an open configuration, respectively). Second distal seal 334 can be distal to first distal seal 332. In some configurations, second distal seal 334 can be a most distal seal of one or more annular seals located along the valve stem 316. First distal seal 332 may be proximal to second distal seal 334 but still distal of proximal aperture 324. First distal seal 332 may be distal to the air inlet C and may be proximal to the water outlet B when the movable portion is in both the first configuration and the second configuration (e.g., when the lumen seal is configured in a closed configuration and an open configuration, respectively).

Valve stem 316 may also have disposed on it a proximal seal 342. Proximal seal 342 may have any of the properties of distal seals 332, 334. For example, proximal seal 342 may be an elastomeric O-ring and may be disposed in an annular circumferential groove or indentation of valve stem 316. A durometer value and outer diameter of proximal seal 342 may be such that the proximal seal 342 has an interference fit with surface 338 (see FIGS. 3A-3B) when valve 310 is inserted in endoscope valve cylinder 339. The interference fit may be loose enough so that valve stem 316 may slidably move relative to endoscope valve cylinder surface 338 but tight enough so that fluids cannot flow longitudinally between a radially outermost surface of proximal seal 342 and surface 338. Proximal seal 342 may have a larger inner diameter than distal seals 332, 334 due to a small diameter of the groove within valve stem 316 at a location of proximal seal 342. Proximal seal 342 may have a larger outer diameter than distal seals 332, 334 due to a wider space defined by surface 338 at the location of seal 342 compared to a space defined by surface 338 at the location of seals 332, 334. It is understood that seals 332, 334 and 342 may be any size to fit around the valve stem 316 and to seal against surface 338 to selectively prevent fluid flow.

Valve stem 316 may also be fitted with a one-way seal 362, which may be disposed longitudinally along the valve stem 316 between first distal seal 332 and proximal seal 342. One-way seal 362 may be formed of an elastomeric material, which may stretch to fit over valve stem 316. One-way seal 362 may be disposed in a groove or indentation of valve stem 316. An inner surface of one-way seal 362 may be sized so that there is a slight interference between an external surface of valve stem 316 and the inner surface of one-way seal 362, so that a tight seal is formed. An outer diameter of one-way seal 362 may be sized so as to form a slight interference fit with surface 338 (see FIGS. 3A-3B). A thin flap of one-way seal 362 may extend radially outward from valve stem 316 at an angle transverse to a longitudinal axis of valve stem 316. For example, the thin flap may extend at an angle between approximately 10 degrees and 80 degrees relative to a longitudinal axis of valve stem 316. The flap of one-way seal 362 may be expandable so that when fluid (e.g., water or air) moves in a distal direction, a positive pressure will expand the flap, maintaining a seal between one-way seal 362 and surface 338 (see FIGS. 3A-3B). Fluid moving proximally will also create a positive pressure, but the positive pressure will produce a force normal to a longitudinal axis of valve stem 316 to radially compress the flap of one-way seal 362 toward valve stem 316. Thus, fluid (e.g., air or water) is permitted to move proximally past one-way seal 362, via an opening formed between one-way seal 362 and surface 338.

Cap 318 may have a stationary portion 370 and a movable portion 372. Although movable portion 372 is described herein as being separate from valve stem 316, it will be appreciated that movable portion 372 and valve stem 316 could be formed of a single integral piece. Stationary portion 370 may remain stationary with respect to valve cylinder 339 when valve 310 is inserted in valve cylinder 339. Stationary portion 370 may include an inner cylindrical member 374 and an outer cylindrical member 376. As shown in FIGS. 3A-3B, inner cylindrical member 374 and outer cylindrical member 376 may be made from a single, unitary piece of material, which may facilitate manufacturing efficiencies. Alternatively, inner cylindrical member 374 and outer cylindrical member 376 may be two separate pieces that are assembled together. Outer cylindrical member 376 may include one or more mating features 378 for mating cap 318 with an outer portion of valve cylinder 339. For example, mating feature 378 may be a protrusion extending radially inward and matable with a corresponding groove or indentation of valve cylinder 339. A distal surface of inner cylindrical member 374 may rest upon a proximal outer surface of valve cylinder 339. A cross-section of inner cylindrical member 374 may be “L” shaped, forming a seat, for a spring (to be described, below).

Movable portion 372 may be proximally and distally (axially or longitudinally) movable relative to valve stem 316 and/or stationary portion 370. Movable portion 372 may be affixed to valve stem 316, so that proximal or distal (axial) movement of movable portion 372 also causes the same motion of valve stem 316. As discussed above, movable portion 372 may be integrally formed with valve stem 316. Movable portion 372 of cap 318 may have a button shape or any other suitable shape. A rim 380 of movable portion 372 may extend in a longitudinal direction between outer cylindrical member 376 and inner cylindrical member 374.

Cap 318 may be fitted with a spring 382, which may be a biasing member. Spring 382 may be a coil spring, leaf spring, or another type of resilient member, such as any member having shape-memory properties. Spring 382 may be, for example, a compression spring. Spring 382 may be configured in cap 318 so that, when spring 382 is in a relaxed state, valve 310 has a first configuration relative to stationary portion 370. When movable portion 372 is moved distally so that valve 310 has a second configuration, spring 382 may be in a deformed, compressed state and may store potential energy due to the deformation (e.g., compression) of spring 382. Spring 382 may have properties, including a stiffness, such that spring 382 exerts a return force (e.g., a known return force) on movable portion 372 after it has been moved distally from the first configuration to the second configuration.

A cap seal 390 may be disposed between movable portion 372 and stationary portion 370. Cap seal 390 may be, for example, an O-ring seal, a washer, or other type of structure and may be formed of elastomeric material. Alternatively, something other than a seal that provides a resistive or frictional force between movable portion 372 and stationary portion 370 may be used in place of cap seal 390. For example, instead of cap seal 390, portions of movable portion 372 or stationary portion 370 may be textured or may have structures or substances disposed thereon that increase resistance between them. As shown in FIGS. 3A and 3B, cap seal 390 may be fixed to movable portion 372, in an annular groove within movable portion 372, and cap seal 390 is movable with respect to stationary portion 370. Alternatively, cap seal 390 may be fixed to stationary portion 370 and movable with respect to movable portion 372.

Cap seal 390 may provide a frictional force between an outer surface of cap seal 390 and an inner surface of stationary portion 370. Thus, when valve 310 is in the second configuration, and spring 382 is in a deformed, compressed state, friction caused by cap seal 390 may resist a return force of spring 382 that urges movable portion 372 and valve stem 316 proximally to the first configuration, in which spring 382 is relaxed. The relationship between a frictional force caused by cap seal 390 and a return force caused by spring 382 may be such that valve 310 automatically moves from the second configuration to the first configuration in a set, predetermined amount of time. In other words, the frictional force may delay the return of valve 310 to the first configuration. The delay may align with a time desired to flush an air channel of an endoscope, as discussed below. However, in some configurations movable portion 372 and stationary portion 370 can be configured in a click-pen type configuration. In such instances, actuation of movable portion 372, as mentioned, can cause the valve stem 316 to translate longitudinally to selectively permit fluid to flow through a lumen of the valve stem 316, as detailed herein.

A stem seal 391 may be disposed between stationary portion 370 and valve stem 316. Stem seal 391 may be, for example, an O-ring seal, a washer, or other type of structure and may be formed of elastomeric material. As shown in FIGS. 3A and 3B, stem seal 391 may be fixed to stationary portion 370, in an annular groove within stationary portion 370, and stem seal 391 is movable with respect to valve stem 316. Alternatively, stem seal 391 may be fixed to valve stem 316 and movable with respect to stationary portion 370. Stem seal 391 may be configured such that fluids (e.g., air or water) cannot pass proximally or distally of stem seal 391.

FIG. 3A shows valve 310 in a first configuration, in which air is flushed through both a water channel and an air channel of an endoscope. In the first configuration, spring 382 may be in a relaxed state, and movable portion 372 may be in a raised position, as a result. In the first configuration, second distal seal 334 may be positioned proximal to a water inlet A of endoscope valve cylinder 339 and also distal to a water outlet B of endoscope valve cylinder 339. First distal seal 332 may be proximal to water outlet B but distal to air inlet C. One-way seal 362 may be proximal to air inlet C and distal to air outlet D.

Thus, in the first configuration, water, or other fluid, from water inlet A may not move proximally past second distal seal 334 and may thus not move to water outlet B. Air, or other fluid, from air inlet C may not move distally along an outer surface of valve stem 316 due to first distal seal 332. However, air from air inlet C may move proximally past one-way seal 362. Air may thus pass into air outlet D. The first configuration may be used after flushing an air channel of an endoscope to ensure that water is removed from the air channel and the water channel before the scope is subject to further reprocessing.

FIG. 3B shows valve 310 in a second, compressed configuration, in which water is flushed down the air channel. Spring 382 may be compressed in the second configuration so that movable portion 372 is translated distally relative to the first configuration. An entirety of valve stem 316 can be shifted distally by a same amount by which movable portion 372 is shifted. Proximal seal 342 may remain proximal to air outlet D. One-way seal 362 may be shifted distally relative to the first configuration, so that air or other fluid from air inlet C may not move past one-way seal 362 (e.g., fluid flow is prevented) because a distal portion of one-way seal 362 fits in a narrowed, tapered region of endoscope valve cylinder 339 so that air cannot flow proximally past the distal portion of one-way seal 362 to reach the proximal movable flap portion of one-way seal 362. As shown in FIGS. 3A and 3B, a first sealing member such as seal 332 can be disposed circumferentially around the valve stem 316 and can be configured to be disposed distal to an air inlet C and an air outlet D and is proximal to liquid outlet B and a liquid inlet A when cap 318 is in the first configuration and the second configuration. Similarly, as illustrated in FIGS. 3A-3B, a second sealing member such as the second seal 334 can be disposed circumferentially around the valve stem and can be disposed distal to the air inlet C, the air outlet D, and the liquid outlet B and proximal to the liquid inlet A of the cleaning valve 310 when the cap 318 is in each of the first configuration and the second configuration.

In the second configuration, second distal seal 334 may be proximal to water inlet A, and first distal seal 332 may remain proximal to water inlet A. Therefore, water or other fluid from water inlet A may enter distally of second distal seal 334 and therefore may not move proximally past second distal seal 334 along an outer surface of valve stem 316. However, water or other fluid may enter distal aperture 326 and travel through lumen 322 and through proximal aperture 324. After water or other fluid exits proximal aperture 324, the water may not flow distally past one-way seal 362 or proximally past the proximal seal 342. However, water or other fluid may flow out air outlet D to flush out the air channel of an endoscope.

If an operator releases movable portion 372 of cap 318 after transitioning valve 310 from the first configuration (FIG. 3A) to the second configuration (FIG. 3B), valve 310 in some configurations, will slowly move back to the first configuration due to restorative forces exerted by spring 382. Hence, valve 310 may not immediately return back to the first configuration due to frictional forces caused by cap seal 390. For example, cap seal 390 may exert forces opposite forces exerted by spring 382, thereby delaying relaxation of the spring 382 and return to the first configuration of the valve 310. However, in some configurations the valve may substantially immediately return back to the first configuration e.g., when cap 318 and/or movable portion 372 are configured in a click-pen type configuration. In any case, valve 310 may continue to deliver water or other fluid to air outlet D until an actuation mechanism 315 is no longer in contact with a lumen seal 311, as detailed herein. For instance, valve 310 may deliver water or other fluid to air outlet D for a predetermined amount of time, which may be specified by a cleaning protocol, without a user pressing on movable portion 372 of cap 318. For example, cap seal 390 and spring 382 may be calibrated so as to flush an air channel for a particular, predetermined amount of time.

After a procedure using an endoscope is completed, an operator may remove an air/water valve used during the procedure from valve cylinder 339. The operator may then insert valve 310 into valve cylinder 339. Cap 318 may be secured to valve cylinder 339 using mating feature 378.

Valve 310 may be inserted into valve cylinder 339 in the first configuration of valve 310. An operator may press down movable portion 372, which compresses spring 382, and shifts valve stem 316 downward, relative to valve cylinder 339 and stationary portion 370. The user may then release movable portion 372 and, configurations with a predetermined cap return time, may attend to other aspects of a post-operative procedure. Even without operator intervention, valve 310 may be maintained in the second, compressed configuration. For instance, the valve 310 may be maintained in the second, compressed configuration until a subsequent actuation of cap 318 (e.g., in configurations where the cap is configured as a click-pen type mechanism) or may be maintained in the second, compressed configuration for a predetermined amount of time (e.g., thirty seconds) so as to flush water through an air channel of the endoscope, thereby removing debris from the air channel. As discussed above, interactions between spring 382 and cap seal 390 may facilitate automatically flushing water for a predetermined amount of time or until a subsequent actuation of the cap 318. Following completion of flushing of water through the air channel, valve 310 may be disposed.

The valve 310 may be used as a gas/water valve 140, a suction valve 145, and/or other suitable type of valve in the endoscope system 200 and/or other suitable system. FIGS. 3A and 3B schematically depict cross-section views of the valve 310 positioned as the gas/water valve 140 within the illustrative endoscope system 200 as illustrated in FIG. 2.

As mentioned, a lumen seal 311 can be coupled to a distal end of the valve stem 316. Valve stem 316 may be coupled to a lumen seal 311 in any suitable manner. Lumen seal 311 can be coupled to or can form the distal end of valve stem 316. In some cases, a portion (e.g., a distal portion) of valve stem 316 proximate or adjacent to distal aperture 326 may be coupled to lumen seal 311. The distal aperture 326 can be selectively in fluid communication with the liquid inlet A. For instance, lumen seal 311 can be configured to fluidically seal distal aperture 326 when the cap 318 and movable portion 372 are in the first configuration and can permit fluid flow into the distal aperture 326 when the cap 318 and movable portion 372 are in the second configuration.

In some configurations, lumen seal 311 can be a flexible lumen seal that is configured to deform radially (traverse to the longitudinal axis of the valve 310) between the first configuration and the second configuration. For instance, the lumen seal 311 can be formed of an elastomer such as silicon rubber, ethylene propylene diene monomer, nitrile rubber, a fluoroelastomer, natural rubber, polyurethane, a thermoplastic such as a thermoplastic elastomer, a thermoplastic polyurethane, or any combination thereof.

Lumen seal 311 can have a radial diameter (transverse to a longitudinal axis of the valve 310) that is bigger than a radial diameter of the distal aperture 326. A central longitudinal axis of lumen seal 311 can be located along the same axis or be coplanar with a central longitudinal axis of the lumen 322. In some configurations, lumen seal 311 can be an individual seal such as an individual monolithic seal. In some configurations, lumen seal 311 can be a one-way seal such as a duck-bill check valve or other type of one-way seal or check valve. For instance, the one-way seal or check valve can be configured to selectively permit fluid flow proximally along lumen 322, as detailed herein. The lumen seal 311 can be a tapered lumen seal having a first diameter at a proximal end of the tapered lumen seal that is less than a second diameter at a distal end of the tapered lumen seal. Employing a tapered (e.g., conical) lumen seal such as a duck-bill check valve an promote aspects herein such as promoting selective fluid flow through lumen seal 311 into the lumen 322 when the lumen seal 311 is in contact with an actuation mechanism 315.

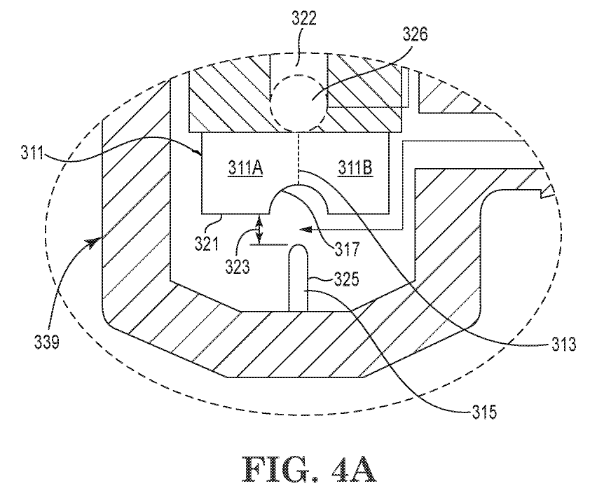

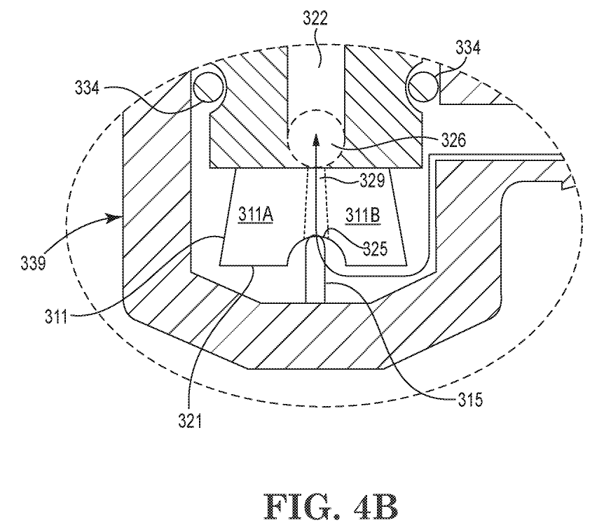

That is, actuation mechanism 315 can be configured to contact the lumen seal to cause the lumen seal 311 to permit fluid flow into the distal aperture 326 (and thereby the lumen 322) when cap 318 and movable portion 372 are in the second configuration. For instance, actuation mechanism 315 can be manifested as an individual substantially longitudinally extending protrusion, as illustrated in FIGS. 3A-3B and 4A-4B. For example, actuation mechanism 315 can extend from a portion of an inner surface of the valve 310 that is distal to the distal end of the valve stem 316. Actuation mechanism 315 can be coplanar with or extending along a common axis with lumen seal 311 and lumen 322, as illustrated in FIGS. 3A-3B and 4A-4B. A proximal end of actuation mechanism 315 can be rounded or curved. However, in some configurations a shape or size of actuation mechanism 315 can be varied, for instance, such that the proximal end of the actuation mechanism 315 is pointed or tapered. In some configurations, actuation mechanism 315 can be manifested as a substantially cylindrical actuation mechanism such as a substantially cylindrical actuation mechanism having a curved proximal end, as illustrated in FIGS. 3A-3B and 4A-4B.

FIGS. 4A-4B show views of a distal end portion 327of the cleaning valve 310 in the first configuration and the second configuration, respectively. Specifically, FIG. 4A shows a magnified view of the distal end portion 327 of the cleaning valve 310 when cap 318 and or movable portion 372 are in the first configuration, as illustrated in FIG. 3A. FIG. 4B shows a magnified view of the distal end portion 327 of the cleaning valve 310 when cap 318 e.g., movable portion 372 is in the second configuration, as illustrated in FIG. 3B.

As illustrated in FIG. 4A, actuation mechanism 315 is distal to the valve stem 316 and lumen seal 311. Stated differently, the actuation mechanism 315 is configured (e.g., has a given longitudinal length, etc.) to be spaced a distance 323 away from distal surface 321 of lumen seal 311 when the movable portion is in the first configuration (e.g., as illustrated in FIG. 3A). As such, lumen seal 311 is in a closed configuration and thereby fluidically seals distal aperture 326 of lumen 322, as illustrated in FIG. 4A. For instance, a first portion 311A and a second portion 311B that comprise lumen seal 311 may be in direct contact and form a fluid tight longitudinally extending interface or slit 313 between first portion 311A and second portion 311B. In some configurations, first portion 311A and second portion 311B may correspond to respective projections forming a duck-bill check valve or other type of one-way seal.

As illustrated in FIG. 4B, actuation mechanism 315 is in contact with and proximal to at least a portion of lumen seal 311. For instance, actuation mechanism 315 can be disposed within the longitudinally extending interface or slit 313 (depicted in FIG. 4A) formed between first portion 311A and second portion 311B of lumen seal 311. When disposed at least partially in slit 313, actuation mechanism 315 can exert a radial force (e.g., opposing radial forces) on lumen seal 311. Responsive to the radial force, slit 313 can radially expand (e.g., in a circumferential manner) to create a fluid flow path or channel 329 that extends entirely through lumen seal 311 (e.g., annularly about at least a portion of an exterior surface of the actuation mechanism 315) and thereby places distal aperture in fluid communication with water inlet A, as illustrated in FIG. 4B. As such, water can flow from the water inlet A into through lumen 322 (from the distal aperture 326 to proximal aperture) and can exit the air outlet D. Removal of actuation mechanism 315 via the proximal movement of valve stem 316 and the cap 318 causes lumen seal 311 to return to the closed configuration (e.g., to return to the first configuration), as illustrated in FIGS. 3A and 4A.

The components of the valve 310 may be formed in any suitable manner. For example, all or at least two or more of the components or portions of valve 310 may be formed monolithically of a single material, one or more components or portions of valve 310 may be formed separate from forming one or more other components or portions of valve 310, one or more components or portions of valve 310 may be embedded in one or more other components or portions of valve 310, and/or one or more components or portions of valve 310 may be formed in one or more other suitable manners. Example suitable coupling mechanisms include, but are not limited to, adhesive, a threaded connection, a luer lock connection, a snap connection, a ball-detent connector, a friction fit, and/or additional or alternative coupling mechanisms.

Any suitable techniques may be utilized to form the components or portions of the components herein. In some examples, though not required, the components or portions of the components herein may be formed using one or more of a molding process, an injection molding process, an over molding process, a casting process, a finishing process, sanding, and/or by or with one or more additional or alternative manufacturing techniques. In one example, the components or portions of the components may be formed using an injection molding process.

The valve 310 may include any suitable number of seals including those described herein. The seals may have any suitable configuration.

The components of the valve 310 may be formed from any suitable materials (e.g., the same or different materials). For example, the components herein may be formed from material including, but not limited to, metals, polymer, plastic, bioplastic, recyclable materials, acrylonitrile butadiene styrene (ABS), polycarbonate, thermoplastic elastomers (TPE), thermoplastic polyurethane (TPU), liquid silicone rubber (LSR), steel, aluminum, and/or other suitable material. In some examples, the seals herein may be formed from one or more of a polymer, thermoplastic elastomers (TPE), thermoplastic polyurethane (TPU), liquid silicone rubber (LSR), and/or other suitable materials. The material of the seals herein may have any suitable durometer.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A medical valve comprising:
a valve stem having a distal end, a proximal end, and a lumen extending along a longitudinal axis of the valve stem between a distal aperture and a proximal aperture; and
a cap including:
a stationary portion; and
a movable portion which is movable relative to the stationary portion and fixed relative to the valve stem and is configured to move between a first configuration and a second configuration; and
a lumen seal coupled to the valve stem, wherein the lumen seal is configured to:
fluidically seal the distal aperture when the movable portion is in the first configuration; and
permit fluid flow into the distal aperture when the movable portion is in the second configuration.

2. The medical valve of claim 1, wherein the lumen seal is a flexible lumen seal that is configured to deform radially between the first configuration and the second configuration.

3. The medical valve of claim 1, wherein the lumen seal is an individual lumen seal.

4. The medical valve of claim 1, wherein the lumen seal is a one-way lumen seal.

5. The medical valve of claim 1, wherein the lumen seal has a radial diameter that is larger than a radial diameter of the distal aperture.

6. The medical valve of claim 1, wherein the lumen seal is a tapered lumen seal having a first diameter at a proximal end of the tapered lumen seal that is less than a second diameter at a distal end of the tapered lumen seal.

7. The medical valve of claim 1, wherein a longitudinal axis of the lumen seal is coplanar with a longitudinal axis of the lumen of the valve stem.

8. The medical valve of claim 1, wherein the lumen seal is a duck-bill check valve.

9. The medical valve of claim 1, including an actuation mechanism that is configured to contact the lumen seal to cause the lumen seal to permit fluid flow into the distal aperture when the cap is in the second configuration.

10. The medical valve of claim 9, wherein the actuation mechanism is an individual substantially longitudinally extending protrusion.

11. The medical valve of claim 9, wherein the actuation mechanism extends from a portion of an inner surface of the medical valve that is distal to the distal end of the valve stem.

12. The medical valve of claim 9, wherein the actuation mechanism is a substantially cylindrical actuation mechanism.

13. The medical valve of claim 12, wherein the actuation mechanism has a rounded proximal end.

14. The medical valve of claim 1, including a first sealing member disposed circumferentially around the valve stem, wherein the first sealing member is configured to be disposed distal to an air inlet and an air outlet and is proximal to liquid outlet and a liquid inlet when the movable portion is in the first configuration and the second configuration.

15. The medical valve of claim 1, including a second sealing member disposed circumferentially around the valve stem, wherein the second sealing member is configured to be disposed distal to an air inlet, an air outlet, and a liquid outlet and be disposed proximal to a liquid inlet of the medical valve when the movable portion is in each of the first configuration and the second configuration.

16. A medical valve comprising:
a valve stem having a distal end, a proximal end, and a lumen extending along a longitudinal axis of the valve stem between a distal aperture and a proximal aperture, wherein the distal aperture is located at the distal end of the valve stem;
a cap including:
a stationary portion;
a movable portion which is movable relative to the stationary portion and fixed relative to the valve stem, wherein the movable portion is configured to move the valve stem between a first configuration and a second configuration;
a seal disposed between the stationary portion and the movable portion; and
a biasing member configured to bias the movable portion to the first configuration; and
a lumen seal coupled to the distal end of the valve stem, wherein the lumen seal is configured to:
fluidically seal the distal aperture when the movable portion is in the first configuration; and
permit fluid flow into the distal aperture when the movable portion is in the second configuration.

17. The medical valve of claim 16, further comprising an actuation mechanism that extends substantially longitudinally from a portion of an inner surface of the medical valve that is distal to the distal end of the valve stem.

18. The medical valve of claim 17, further comprising:
a first sealing member disposed circumferentially around the valve stem, wherein the first sealing member is configured to be disposed distal to an air inlet and an air outlet and is proximal to liquid outlet and a liquid inlet when the movable portion is in the first configuration and the second configuration; and
a second sealing member disposed circumferentially around the valve stem, wherein the second sealing member is configured to be disposed distal to the air inlet, the air outlet, and the liquid outlet and proximal to the liquid inlet of the medical valve when the movable portion is in each of the first configuration and the second configuration.

19. A medical valve comprising:
a valve body;
a valve stem disposed in the valve body, the valve stem having a distal end, a proximal end, and a lumen extending along a longitudinal axis of the valve stem between a distal aperture and a proximal aperture, wherein the distal aperture is located at the distal end of the valve stem, wherein the valve stem is configured to translate within the valve body; and
a cap including:
a stationary portion;
a movable portion which is movable relative to the stationary portion and fixed relative to the valve stem, wherein the movable portion is configured to move between a first configuration and a second configuration;
a seal disposed between the stationary portion and the movable portion; and
a biasing member configured to bias the movable portion to the first configuration;
a lumen seal coupled to the valve stem, wherein the lumen seal is configured to:
fluidically seal the distal aperture when the movable portion is in the first configuration; and
permit fluid flow into the distal aperture when the movable portion is in the second configuration; and
an actuation mechanism that is configured to:
contact a distal surface of the lumen seal to cause the lumen seal to deform and permit the fluid flow into the distal aperture when the movable portion is in the second configuration.

20. The medical valve of claim 19, wherein the actuation mechanism is configured to be spaced a distance away from the distal surface of the lumen seal when the movable portion is in the first configuration.
